(19) United States
(12) Patent Application Publication
(10) Pub. No.: US 2025/0200748 A1
(43) Pub. Date: Jun. 19, 2025

(54) MULTIMODAL FOUNDATION MODEL FOR PATHOLOGY ANALYSIS

(71) Applicant: THE BRIGHAM AND WOMEN'S HOSPITAL, INC., Boston, MA (US)

(72) Inventors: Faisal Mahmood, Brookline, MA (US); Ming-Yang Lu, Cambridge, MA (US); Richard Chen, Gaithersburg, MD (US); Bowen Chen, Stoneham, MA (US)

(21) Appl. No.: 18/982,577

(22) Filed: Dec. 16, 2024

Related U.S. Application Data
(60) Provisional application No. 63/610,645, filed on Dec. 15, 2023.
(60) Provisional application No. 63/611,059, filed on Dec. 15, 2023.

(51) Int. Cl. G06T 7/00; G06T 7/11; G06V 10/26; G06V 10/74; G06V 10/764; G06V 10/774; G06V 10/776; G06V 10/80; G16H 50/50; G16H 70/60

(52) U.S. Cl. CPC G06T 7/0012; G06T 7/11; G06V 10/26; G06V 10/761; G06V 10/764; G06V 10/7747; G06V 10/776; G06V 10/809; G16H 50/50; G16H 70/60; G06T 2207/20021; G06T 2207/20081; G06T 2207/30096

(57) ABSTRACT

Systems and methods are provided for analysis of pathology data. Either a input data representing a pathology or a search query is received as an input and a first set of tokens is generated from the one of the input data representing a pathology and the search query from the input. The first set of tokens is matched to a second set of tokens at a multimodal fusion model trained on a pretraining dataset complied from a plurality of pathology-related sources. An output is provided based on the second set of tokens.

### RELATED APPLICATIONS

This application claims priority from each of U.S. Provisional Application No. 63/610,645, filed 15 Dec. 2024 and entitled “A GENERALIST VISION-LANGUAGE MODEL FOR COMPUTATIONAL PATHOLOGY,” and U.S. Provisional Application No. 63/611,059, filed 15 Dec. 2024 and entitled “A GENERALIST SELF-SUPERVISED VISION MODEL FOR COMPUTATIONAL PATHOLOGY.” The subject matter of each of these applications is hereby incorporated by reference in its entirety.

### TECHNICAL FIELD

This invention relates to clinical decision support systems, and more particularly, to a multimodal foundation model for pathology analysis.

### BACKGROUND

The gold standard for the diagnosis of many diseases remains the examination of tissue by a pathologist. Computational pathology, which leverages artificial intelligence (AI) to solve problems in pathology, has demonstrated considerable advances across many tasks, including metastasis detection, cancer subtyping, survival prediction, unknown primary origin site prediction, image search, and mutation prediction, among other tasks. Additionally, current strides in the field are made under the paradigm of developing models targeting specific tasks using large cohorts of labeled training examples, such as in lymph node metastasis detection and prostate cancer grading.

Unfortunately, the process of data collection and annotation of whole slide images (WSIs) is labor-intensive and is not scalable to open-set recognition problems or rare diseases, both of which are common to the practice of pathology. With thousands of possible diagnoses and many other tasks, training separate models for every step of the pathology workflow is untenable. Additionally, as diverse as these tasks are, they are all analyses of visual data or include other structured information such as “omics” and other multimodal data sources. However, the practice of pathology and the communication of pathological findings make extensive use of natural language, be it in the form of the report that the pathologist prepares for the patient and their treating clinician, the journal article that details a new histopathologic entity, the textbook chapter that teaches residents how to practice pathology, or the reporting of molecular testing, IHC testing, or molecular measurements written as natural language.

### SUMMARY

In accordance with one example, a system is provided. The system includes a processor and a non-transitory computer readable medium storing instructions executable by the processor. The machine-executable instructions include a first encoder that reduces a received set of pathology data to a first set of tokens and a multimodal fusion model that matches the first set of tokens to a second set of tokens. The multimodal fusion model is trained on a pretraining dataset of pathology data and data characterizing the pathology data complied from a plurality of pathology-related sources, with a given training sample comprising pathology data and data characterizing the pathology data. A user interface displays an output representing the second set of tokens.

In accordance with another example, a method is provided. Either an input representing a pathology or a search query is received as an input and a first set of tokens is generated from one of the input representing the pathology and the search query from the input. The first set of tokens is matched to a second set of tokens at a multimodal fusion model trained on a pretraining dataset complied from a plurality of pathology-related sources. A given training sample includes a set of pathology data and text describing the pathology data. An output is provided based on the second set of tokens.

In accordance with a further example, a system is provided. The system includes a processor and a non-transitory computer readable medium storing instructions executable by the processor. The machine-executable instructions include a text encoder that reduces a received search to a set of text tokens and a multimodal fusion model that matches the set of text tokens to a set of visual tokens. The multimodal fusion model is trained on a pretraining dataset complied from a plurality of pathology-related sources, with a given training sample within the pretraining dataset comprising a pathology image and text describing the image. A user interface that displays an image associated with a set of visual tokens.

### BRIEF DESCRIPTION OF THE DRAWINGS

FIG. 1 illustrates an example of a system implementing a multimodal foundation model for human pathology analysis;

FIG. 2 illustrates an example of a system using a multimodal foundation model for cross-modal search;

FIG. 3 illustrates a system for training a multimodal fusion model;

FIG. 4 illustrates one example of a method for analysis of pathology data and text;

FIG. 5 illustrates one example of a method for analysis of a pathology image;

FIG. 6 illustrates one example of a method for cross-modal search of pathology images; and

FIG. 7 is a schematic block diagram illustrating an exemplary system of hardware components capable of implementing examples of the systems and methods disclosed herein.

### DEFINITIONS

“Pathology data”, as used herein, represents a set of data representing a pathology. A pathology image is an example of pathology data.

A “pathology image,” as used herein, is an image of tissue from a human body used for the diagnosis of disease. Non-exclusive examples of pathology images include hematoxylin and eosin stain images, other images acquired using histological stains, immunochemistry images, electron microscope images, cytology images, multiplex images, gross pathology images, radiological images, and any other complimentary images.

A “pathology-related source” is numerical or text data that describes the pathology image, and can include educational articles, image captions, reporting of molecular and IHC testing in a pathology report, pathology case reports, molecular measurements obtained from the same spatial location of the tissue from which the pathology image is acquired, and other pathology images corresponding to the same image collected with different histological staining and image acquisition techniques.

### DETAILED DESCRIPTION

The systems and methods described herein provide a multimodal foundation model developed using diverse sources of histopathology images, biomedical text, and image-caption pairs via task-agnostic pretraining. The system includes utilizes a first encoder, that encodes data representing a pathology, a second encoder, that encodes information that characterizes the pathology, and a multimodal fusion model, and is trained via a combination of self-supervised learning objectives that seek to align the two modalities in the model's representation space and a objective that learns to predict characterizing data corresponding to a set of pathology data. The system can be applied to a wide range of downstream tasks involving either or both histopathology images and text, achieving state-of-the-art performance on histology image classification, segmentation, captioning, text-to-image, and image-to-text retrieval. The system represents a substantial leap over concurrent visual-language pretrained systems for histopathology, with the potential to directly facilitate a wide array of machine learning-based workflows requiring minimal or no further supervised fine-tuning.

FIG. 1 illustrates an example of a system 100 implementing a multimodal foundation model for human pathology analysis. The system 100 includes a processor 102, an output device 104, such as a display, and a non-transitory computer readable medium 110 storing executable instructions, executed by the processor 102. It will be appreciated that the executable instructions can be spread across multiple non-transitory computer readable media that are operatively connected via an appropriate data connection, such that the executable instructions can be executed by multiple processors. In particular, the system 100 can be trained across multiple graphics processing units, with a number of graphics processing units and storage at the non-transitory computer readable medium 110 used for a given application be scalable with the application and the amount of available training data.

The executable instructions stored on the non-transitory computer readable medium 110 include a data interface 111 that receives data representing a pathology and conditions the data for further processing. In one example, in which the data includes an image, the conditioning process can include placing the image in an appropriate size and format for analysis as well as applying one or more image filters to reduce noise and artifacts. The image is provided to a encoder 112 that converts received data into a lower dimensionality representation as a first set of tokens. In one example, the encoder 112 can be implemented as an artificial neural network that is trained on a corpus of training data, such as a convolutional neural network or an autoencoder. Alternatively, one or more feature extraction algorithms can be used to reduce the data to a smaller set of numbers. In an example in which the data includes an image, the extracted features can include a histogram of ordered gradients, a scale-invariant feature transformation, extraction of local binary patterns, extraction of frequency-based features, and similar algorithms. It will be appreciated that the encoder 112 can operate at the level of the entire dataset or on portion of the dataset. For example, for image segmentation tasks, the data interface 111 may divide the received image into tiles and provide the tiles to the encoder 112 to represent each tile as a set of visual tokens in the first set of tokens.

In one example, the encoder 112 is implemented as a vision encoder that is trained with self-supervised learning based on student-teacher knowledge distillation and masked image modeling. The training method uses, as part of an objective function, a self-distillation loss and a masked image modeling loss. Self-distillation minimizes the predictive categorical distributions from the teacher and student network obtained from two augmented views of the same image by minimizing their cross-entropy loss. The teacher is updated as an exponential moving average of previous iterations of the student. Masked image modeling involves strategically masking or corrupting specific regions within an input image and training the model to predict the masked or corrupted regions based on the remaining contextual information. This approach captures high-level visual features and context. Specifically, two augmented views of an input image are subsequently randomly masked. While the augmented views of the image are propagated through the teacher network, the student network receives the masked image views as inputs. For the self-distillation objective, we compute cross-entropy loss between the token from the teacher network and the token from the student network, with the output of the masked tokens from the student network to predict the patch tokens from the teacher network, where the teacher network can be regarded as an online tokenizer.

In one example, the encoder 112 can be trained with self-supervised learning based on contrastive learning. The training method uses, as part of an objective function, a contrastive learning loss that encourages representations of similar pathology data (positive pairs) to be closer in the embedding space while pushing representations of dissimilar pathology data (negative pairs) further apart. Where the pathology data is an image, the method can involve generating multiple augmented views of the same image to form positive pairs, while augmented views of different images are treated as negative pairs.

In another example, the encoder 112 can be trained with self-supervised learning based on a reconstruction objective without student-teacher knowledge distillation. The training method, uses, as part of the objective function, a reconstruction loss whereby learning to construct the data allows the model to learn a high-quality representation. A reconstruction loss involves strategically masking or corrupting specific portions of the data, such as regions within an input image, and training the model to predict the masked or corrupted portions based on the remaining contextual information. A mean-squared error or cross-entropy loss is used to measure the alignment between the predicted tokens and the ground truth tokens, encouraging the model to capture rich contextual and semantic features. The masking strategy ensures that the encoder 112 learns to infer missing information while understanding the global structure of the input data.

The first set of tokens is provided to a multimodal fusion model 114 that matches the first set of I tokens to a second set of tokens characterizing pathology data paired with sets of tokens representing the pathology data. In one example, a similarity metric, such as a cosine similarity metric, or a distance metric can be calculated between the first set of tokens and the sets of tokens charactering pathology data, and one or more sets of pathology data with sets of tokens charactering pathology data having a highest similarity or smallest distance can be selected. Alternatively, all pathology data with sets of tokens having a similarity or distance that meets a threshold can be selected. These tokens can represent a label, such as a tissue type or a disorder, associated with each set of pathology data or portion of the pathology data. The tokens can be used for classification of all or portions of the image, segmentation of the image into different tissue types, or generation of a caption or description for the image.

The multimodal fusion model 114 can be trained on a set of pairs of pathology data and data characterizing the pathology data, which are represented in the model as paired sets of data tokens and characerization tokens. For example, the multimodal fusion model 114 can be implemented as a linear projector or a visual abstractor. In the illustrated example, the multimodal fusion model 114 is trained via a combination of contrastive alignment objectives that seek to align two data modalities in a representation space. In one implementation, the self-supervised learning objective may also include a captioning objective that learns to predict the caption corresponding to an image. A user interface 116 provides an output to a user at the output device 104 based on the second of tokens. For example, the output could be a text label for a disorder or tissue type associated with all or a portion of the slide in a classification operation, IHC or molecular testing results associated with the pathology image, gene or protein expression values associated with the pathology image, an image with segmented regions of a given tissue type highlighted for segmentation operations, any other appropriate output for communicating the visual content of the image. In one implementation, in classification tasks, the image can be displayed as a heatmap representing a similarity metric associated with each of a plurality of tiles comprising the image for a text label applied to the overall image.

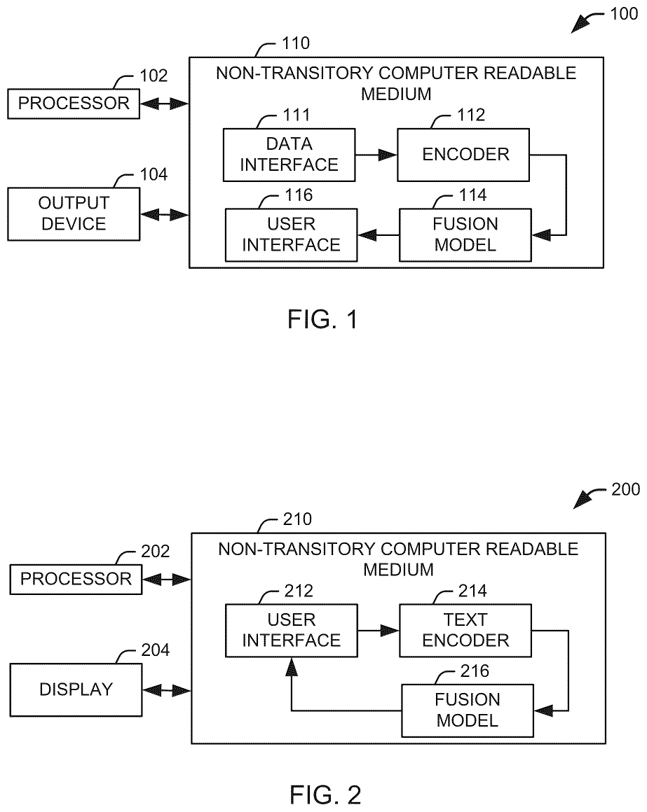

FIG. 2 illustrates an example of a system 200 using a multimodal foundation model for cross-modal search. Specifically, the system 200 allows for images to be retrieved via a text query for a certain condition, tissue type, location within the body, or other characteristics. The system 200 includes a processor 202, a display 204, and a non-transitory computer readable medium 210 storing executable instructions, executed by the processor 202. It will be appreciated that the executable instructions can be spread across multiple non-transitory computer readable media that are operatively connected via an appropriate data connection, such that the executable instructions can be executed by multiple processors. In particular, the system 200 can be trained across multiple graphics processing units, with a number of graphics processing units and storage at the non-transitory computer readable medium 210 used for a given application be scalable with the application and the amount of available training data.

The executable instructions stored on the non-transitory computer readable medium 210 include a user interface 212 that allows a user to enter a search query. A text encoder 214 that reduces the text to a set of tokens that are compatible with an embedding space of a multimodal fusion model 216. The text encoder 214 can use any appropriate tokenization technique including one or more of word-based tokenization, sub-word tokenization, or character-level tokenization. These text tokens can represent a label, such as a tissue type or a disorder, associated with each image or portion of the image.

The sets of text tokens are provided to the multimodal fusion model 216 that match the set of text tokens to sets of visual tokens representing stored images. In one example, a similarity metric, such as a cosine similarity metric, or a distance metric can be calculated between the set of text tokens and each of the sets of visual tokens, and one or more images with sets of visual tokens having a highest similarity or smallest distance can be selected. Alternatively, all images with sets of visual tokens having a similarity or distance that meets a threshold can be selected. The multimodal fusion model 216 can be trained on a set of pairs of images and associated text, which are represented in the model as paired sets of visual tokens and text tokens. In the illustrated example, the multimodal fusion model 216 is trained by first training the vision encoder with student-teacher knowledge distillation and masked imaging modeling objectives on a corpus of pathology image-only data, followed by contrastive alignment objectives that seek to align the image and text modalities in a representation space and a captioning objective that learns to predict the caption corresponding to an image. The user interface 212 provides the selected images to a user at the display 204. It will be appreciated that the model can be trained with a combination of different self-supervised learning objectives.

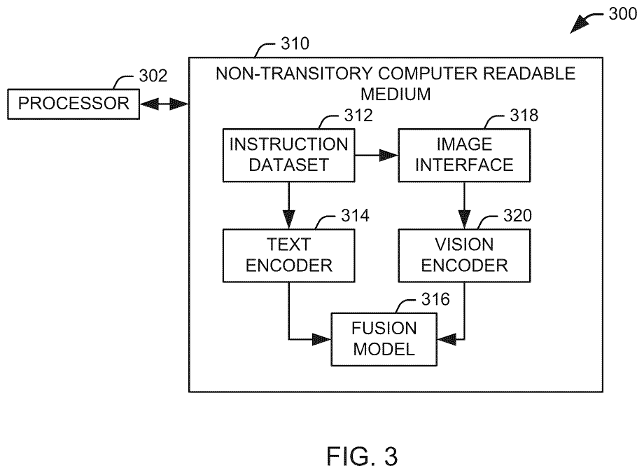

FIG. 3 illustrates a system 300 for training a multimodal fusion model that can be used in the systems of FIG. 1 and FIG. 2. The system 300 includes a processor 302 and a non-transitory computer readable medium 310 storing executable instructions that are executed by the processor 302. It will be appreciated that the executable instructions can be spread across multiple non-transitory computer readable media that are operatively connected via an appropriate data connection, such that the executable instructions can be executed by multiple processors.

The system 300 includes a pretraining dataset 312 complied from a plurality of pathology-related sources. Each training sample in the pretraining dataset includes at least one pathology image and text describing the image or images, generally in the form of an image and an associated caption. In one example, the pretraining dataset can include around four hundred thousand instructions. It will be appreciated, however, that the model can be scaled to utilize more or less training data, with hyperparameters of the model adjusted with the amount of available data, the available hardware, and the application. Data filtering was performed for each source individually to ensure quality and relevance for training a pathology-specific vision language assistant. For example, image captions that were overly short (e.g., <twelve words) or uninformative and overly generic (e.g., “An H&E image of tumor”) were omitted. Captions related to animal pathology, for example, text containing keywords related to animals such as “rat” or “pig” as well as experimental studies, identified as text containing appropriate keywords, such as “experimental” or “positive control” were also omitted. In one implementation, this filtering is performed using regular expression pattern matching process.

The executable instructions include a text encoder 314 that reduces the text for each caption to a set of tokens that are compatible with an embedding space of a multimodal fusion model 316. The text encoder 314 can use any appropriate tokenization technique including one or more of word-based tokenization, sub-word tokenization, or character-level tokenization. An image interface 318 receives an image corresponding to the caption and conditions the image for further processing. The conditioning process can include placing the image in an appropriate size and format for analysis as well as applying one or more image filters to reduce noise and artifacts.

A vision encoder 320 converts each image into a lower dimensionality representation of the image as a set of visual tokens. In one example, the vision encoder 320 can be implemented as an artificial neural network that is trained on a corpus of training images, such as a convolutional neural network or an autoencoder. Alternatively, one or more feature extraction algorithms can be used to reduce the image to a smaller set of numbers, such as a histogram of ordered gradients, a scale-invariant feature transformation, extraction of local binary patterns, extraction of frequency-based features, and similar algorithms. In one example, the vision encoder 320 can comprise a transformer encoder, trained on a corpus of histology images, that operates directly on patches of the image to assign one or more categorical parameters to the image. In one implementation, the vision encoder 320 can use an adaptation of the ViT-Large (ViT-L) architecture.

The multimodal fusion model 316 is trained on the outputs of the text encoder 312 and the vision encoder 320. In the illustrated implementation, the multimodal fusion model 320 is trained using an objective function having a contrastive objective component that aligns the image and text encoders by maximizing the cosine-similarity scores between paired image and text embeddings and a captioning objective that maximizes the likelihood of generating the correct text conditioned on the image and previously generated text. Once the multimodal fusion model 316 is fully trained on the pretraining dataset 312, it can be employed for classification, segmentation, and cross-modal search tasks as described previously.

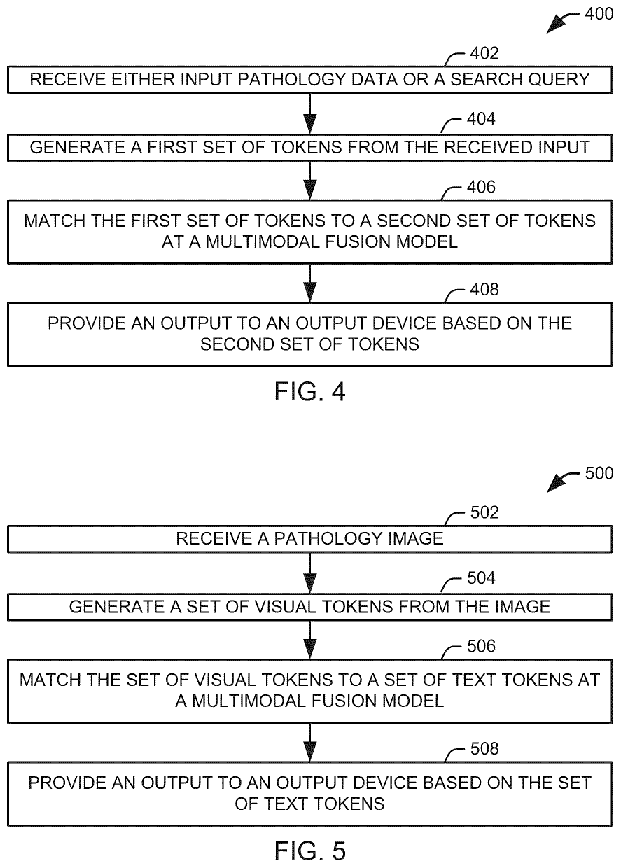

In view of the foregoing structural and functional features described above, methods in accordance with various aspects of the present invention will be better appreciated with reference to FIGS. 4-6. While, for purposes of simplicity of explanation, the methods of FIGS. 4-6 are shown and described as executing serially, it is to be understood and appreciated that the present invention is not limited by the illustrated order, as some aspects could, in accordance with the present invention, occur in different orders and/or concurrently with other aspects from that shown and described herein. Moreover, not all illustrated features may be required to implement a method in accordance with an aspect of the present invention.

FIG. 4 illustrates one example of a method 400 for analysis of pathology data and text. At 402, either an input representing a pathology or a search query is received. At 404, a first set of tokens is generated from the received input. For example, where the input is a search query, the query can be provided to a text encoder to reduce the query to a set of text tokens. Where the input represents a pathology image, the image can be provided to an appropriate encoder to generate a set of tokens. In one example, in which the input is a pathology encoder, the image is provided to a vision encoder trained on a plurality of pathology images via a self-supervising learning algorithm using an objective function including a self-distillation loss and a masked image modeling loss.

Regardless, the generated set of tokens is provided to a multimodal fusion model at 406 to match the tokens to a second set of tokens. The multimodal fusion model is trained on a pretraining dataset complied from a plurality of pathology-related sources, with a given training sample within the pretraining dataset comprising a data representing a pathology and text describing the pathology. Where the input represents a pathology, the multimodal fusion model can match the first set of tokens to a set of text tokens that predict a caption associated with the data, for example, as a set of text tokens having a highest similarity metric in a projection space associated with the multimodal fusion model. Where the input is a text query, the multimodal fusion model can match the text tokens to one or more sets of visual tokens to find data, such as images, responsive to the search. In one example, the multimodal fusion model can compute a similarity metric between the set of text tokens and a plurality of sets of visual tokens associated with the multimodal fusion model and match the set of text tokens with each set of visual tokens for which the similarity metric meets a threshold value or a predetermined number of sets of visual tokens for having the highest similarity metrics.

At 408, an output is provided at an output device, such as a display, via user interface based on the second set of tokens. In one implementation, for an input representing a pathology, the provided output can be a class label associated with the input. In another implementation for an input image, the provided output can include a class label for each of a plurality of tiles associated with the input image. In a further implementation for an input image, the provided output can be a class label associated with the input image and a heat map showing a similarity metric for each of a plurality of tiles associated with the input image given the class label for the image. In a still further implementation for an input image, the provided output is a segmented representation of the input image. In one implementation for a text input, the provided output can be one or more images that are responsive to the search query. The output can also be stored on a non-transitory computer readable medium, for example, as part of a medical record.

FIG. 5 illustrates one example of a method 500 for analysis of a pathology image. At 502, a pathology image is received for analysis. For example, the pathology image can be received from local storage, directly from an imaging system or a storage medium associated with the imaging system, via a local internet connection, for example, from an associated server, or via an Internet connection, such as from an electronic health records system. At 504, a set of visual tokens is generated from the received image at a vision encoder. In one example, the vision encoder is trained on a plurality of pathology images via a self-supervising learning algorithm using an objective function including a self-distillation loss and a masked image modeling loss. In one example, the input image can be divided up into a plurality of tiles, with each tile analyzed separately at a vision encoder to provide a set of visual tokens for each of the plurality of tiles.

The set of visual tokens is provided to a multimodal fusion model at 506 to match the visual tokens to a set of text tokens. The multimodal fusion model is trained on a pretraining dataset complied from a plurality of pathology-related sources, with a given training sample within the pretraining dataset comprising a pathology image and text describing the image. The multimodal fusion model matches the visual tokens to a set of text tokens that predict a caption associated with the image, for example, as a set of text tokens having the highest similarity metric in a projection space associated with the multimodal fusion model. Where the image is divided into tiles, each tile can be analyzed separately to provide either an associated set of text tokens or a similarity metric for a set of text tokens associated with the entire image. At 508, an output is provided at an output device, such as a display, via user interface based on the set of text tokens match at the multimodal fusion model. For example, the provided output can be a class label associated with the input image, a class label for each of a plurality of tiles associated with the input image, a heat map showing a similarity metric for each of a plurality of tiles associated with the input image given a class label for the image, or a segmented representation of the input image. The output can also be stored on a non-transitory computer readable medium, for example, as part of a medical record.

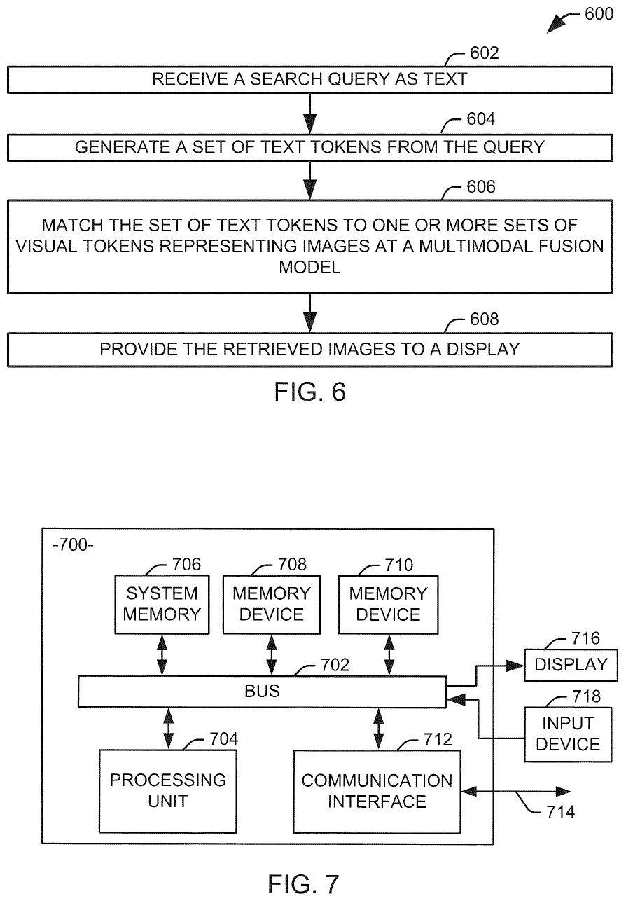

FIG. 6 illustrates one example of a method 600 for cross-modal search of pathology images. At 602, a search query is received as text. For example, the search query can be provided by a user at a local user interface, provided from a remote system via a network interface, or selected from a locally stored batch of search requests. At 604, a set of text tokens is generated from the received query at a text encoder. The generated set of text tokens is provided to a multimodal fusion model at 606 to match the tokens to one or more sets of visual tokens. The multimodal fusion model is trained on a pretraining dataset complied from a plurality of pathology-related sources, with a given training sample within the pretraining dataset comprising a pathology image and text describing the image. The multimodal fusion model can match the text tokens to one or more sets of visual tokens to find images responsive to the search. For example, the multimodal fusion model can compute a similarity metric between the set of text tokens and a plurality of sets of visual tokens associated with the multimodal fusion model and match the set of text tokens with each set of visual tokens for which the similarity metric meets a threshold value or a predetermined number of sets of visual tokens for having the highest similarity metrics. At 608, the retrieved images are provided to a display via a user interface. The retrieved images can also be stored on a non-transitory computer readable medium for later review.

FIG. 7 is a schematic block diagram illustrating an exemplary system 700 of hardware components capable of implementing examples of the systems and methods disclosed herein. The system 700 can include various systems and subsystems. The system 700 can be a personal computer, a laptop computer, a workstation, a computer system, an appliance, an application-specific integrated circuit (ASIC), a server, a server BladeCenter, a server farm, etc.

The system 700 can include a system bus 702, a processing unit 704, a system memory 706, memory devices 708 and 710, a communication interface 712 (e.g., a network interface), a communication link 714, a display 716 (e.g., a video screen), and an input device 718 (e.g., a keyboard, touch screen, and/or a mouse). The system bus 702 can be in communication with the processing unit 704 and the system memory 706. The additional memory devices 708 and 710, such as a hard disk drive, server, standalone database, or other non-volatile memory, can also be in communication with the system bus 702. The system bus 702 interconnects the processing unit 704, the memory devices 706-710, the communication interface 712, the display 716, and the input device 718. In some examples, the system bus 702 also interconnects an additional port (not shown), such as a universal serial bus (USB) port.

The processing unit 704 can be a computing device and can include an application-specific integrated circuit (ASIC). The processing unit 704 executes a set of instructions to implement the operations of examples disclosed herein. The processing unit can include a processing core.

The additional memory devices 706, 708, and 710 can store data, programs, instructions, database queries in text or compiled form, and any other information that may be needed to operate a computer. The memories 706, 708 and 710 can be implemented as computer-readable media (integrated or removable), such as a memory card, disk drive, compact disk (CD), or server accessible over a network. In certain examples, the memories 706, 708 and 710 can include text, images, video, and/or audio, portions of which can be available in formats comprehensible to human beings.

Additionally or alternatively, the system 700 can access an external data source or query source through the communication interface 712, which can communicate with the system bus 702 and the communication link 714.

In operation, the system 700 can be used to implement one or more parts of a system for analysis of pathology images. Computer executable logic for implementing the diagnostic system resides on one or more of the system memory 706, and the memory devices 708 and 710 in accordance with certain examples. The processing unit 704 executes one or more computer executable instructions originating from the system memory 706 and the memory devices 708 and 710. The term “computer readable medium” as used herein refers to a medium that participates in providing instructions to the processing unit 704 for execution. This medium may be distributed across multiple discrete assemblies all operatively connected to a common processor or set of related processors.

Implementation of the techniques, blocks, steps, and means described above can be done in various ways. For example, these techniques, blocks, steps, and means can be implemented in hardware, software, or a combination thereof. For a hardware implementation, the processing units can be implemented within one or more application specific integrated circuits (ASICs), digital signal processors (DSPs), digital signal processing devices (DSPDs), programmable logic devices (PLDs), field programmable gate arrays (FPGAs), processors, controllers, micro-controllers, microprocessors, other electronic units designed to perform the functions described above, and/or a combination thereof.

Also, it is noted that the embodiments can be described as a process which is depicted as a flowchart, a flow diagram, a data flow diagram, a structure diagram, or a block diagram. Although a flowchart can describe the operations as a sequential process, many of the operations can be performed in parallel or concurrently. In addition, the order of the operations can be re-arranged. A process is terminated when its operations are completed but could have additional steps not included in the figure. A process can correspond to a method, a function, a procedure, a subroutine, a subprogram, etc. When a process corresponds to a function, its termination corresponds to a return of the function to the calling function or the main function.

Furthermore, embodiments can be implemented by hardware, software, scripting languages, firmware, middleware, microcode, hardware description languages, and/or any combination thereof. When implemented in software, firmware, middleware, scripting language, and/or microcode, the program code or code segments to perform the necessary tasks can be stored in a machine-readable medium such as a storage medium. A code segment or machine-executable instruction can represent a procedure, a function, a subprogram, a program, a routine, a subroutine, a module, a software package, a script, a class, or any combination of instructions, data structures, and/or program statements. A code segment can be coupled to another code segment or a hardware circuit by passing and/or receiving information, data, arguments, parameters, and/or memory contents. Information, arguments, parameters, data, etc. can be passed, forwarded, or transmitted via any suitable means including memory sharing, message passing, ticket passing, network transmission, etc.

For a firmware and/or software implementation, the methodologies can be implemented with modules (e.g., procedures, functions, and so on) that perform the functions described herein. Any machine-readable medium tangibly embodying instructions can be used in implementing the methodologies described herein. For example, software codes can be stored in a memory. Memory can be implemented within the processor or external to the processor. As used herein the term “memory” refers to any type of long term, short term, volatile, nonvolatile, or other storage medium and is not to be limited to any particular type of memory or number of memories, or type of media upon which memory is stored.

Moreover, as disclosed herein, the term “storage medium” can represent one or more memories for storing data, including read only memory (ROM), random access memory (RAM), magnetic RAM, core memory, magnetic disk storage mediums, optical storage mediums, flash memory devices and/or other machine-readable mediums for storing information. The term “machine-readable medium” includes, but is not limited to, portable or fixed storage devices, optical storage devices, wireless channels, and/or various other storage mediums capable of storing that contain or carry instruction(s) and/or data.

In the preceding description, specific details have been set forth in order to provide a thorough understanding of example implementations of the invention described in the disclosure. However, it will be apparent that various implementations may be practiced without these specific details. For example, circuits, systems, networks, processes, and other components may be shown as components in block diagram form in order not to obscure the example implementations in unnecessary detail. In other instances, well-known circuits, processes, algorithms, structures, and techniques may be shown without unnecessary detail in order to avoid obscuring the examples. The description of the example implementations will provide those skilled in the art with an enabling description for implementing an example of the invention, but it should be understood that various changes may be made in the function and arrangement of elements without departing from the spirit and scope of the invention. Accordingly, the present invention is intended to embrace all such alterations, modifications, and variations that fall within the scope of the appended claims.

What is claimed is:

1. A system comprising:
a processor; and
a non-transitory computer readable medium storing instructions executable by the processor, the machine-executable instructions comprising:
a first encoder that reduces received data representing a pathology to a first set of tokens;
a multimodal fusion model that matches the first set of tokens to a second set of tokens characterizing the pathology, the multimodal fusion model being trained on an pretraining dataset complied from a plurality of pathology-related sources, a given training sample within the pretraining dataset comprising a data representing a pathology and data characterizing the data representing the pathology; and
a user interface that displays an output representing the second set of tokens.

2. The system of claim 1, wherein the received data is an image, the first set of tokens is a set of visual tokens, and the second set of tokens is a set of text tokens.

3. The system of claim 2, further comprising an image interface that receives the received image, divides the received image into a plurality of tiles, and provides the plurality of tiles to the first encoder to provide a set of visual tokens for each of the plurality of tiles.

4. The system of claim 3, wherein the multimodal fusion model provides a set of text tokens for each of the plurality of tiles, the user interface displaying an output representing the sets of text token for each of the plurality of tiles.

5. The system of claim 3, wherein the multimodal fusion model provides a set of text tokens for the received image and a similarity metric for each tile for the set of text tokens, the output representing the similarity metric for each tile.

6. The system of claim 3, wherein the first encoder is trained on a plurality of pathology images via a self-supervising learning algorithm using an objective function including a self-distillation loss and a masked image modeling loss.

7. The system of claim 2, wherein the multimodal fusion model is trained using an objective function having a contrastive objective component that aligns the first and second encoders by maximizing cosine-similarity scores between paired image and text embeddings and a captioning objective that maximizes the likelihood of generating the correct text conditioned on the image and previously generated text.

8. A method comprising:
receiving one of an input representing a pathology and a search query;
generating a first set of tokens from the one of the input representing a pathology and the search query; and
matching the first set of tokens to a second set of tokens at a multimodal fusion model trained on a pretraining dataset complied from a plurality of pathology-related sources, a given training sample within the pretraining dataset comprising a data representing a pathology and text describing the image; and
providing an output based on the second set of tokens.

9. The method of claim 8, wherein the input representing the pathology is an input image.

10. The method of claim 9, wherein the one of the input image and the search query is the input image, and the provided output is a class label associated with the input image.

11. The method of claim 9, wherein the one of the input image and the search query is the input image, and the provided output is a segmented representation of the input image.

12. The method of claim 9, wherein the one of the input representing the pathology and the search query is the search query, and the provided output is an image that is responsive to the search query.

13. The method of claim 9, wherein the one of the input image and the search query is the input image, and generating the first set of tokens comprises providing the input image to a vision encoder trained on a plurality of pathology images via a self-supervising learning algorithm using an objective function including a self-distillation loss and a masked image modeling loss.

14. The method of claim 9, wherein the one of the input image and the search query is the search query matching the first set of tokens to the second set of tokens at the multimodal fusion model comprises computing a similarity metric between the set of text tokens and a plurality of sets of visual tokens associated with the multimodal fusion model and matching the set of text tokens with each set of visual tokens for which the similarity metric meets a threshold value.

15. The method of claim 9, further comprising”
dividing the input image into a plurality of tiles; and
providing the plurality of tiles to a vision encoder to provide a set of visual tokens for each of the plurality of tiles;
wherein matching the first set of tokens to the second set of tokens at the multimodal fusion model comprises matching the set of visual tokens for each of the plurality of tiles with a corresponding set of text tokens, the output being provided according to the set of text tokens for each of the plurality of tiles.

16. The method of claim 9, further comprising”
dividing the input image into a plurality of tiles; and
providing the plurality of tiles to a vision encoder to provide a set of visual tokens for each of the plurality of tiles;
wherein matching the first set of tokens to the second set of tokens at the multimodal fusion model comprises generating a similarity metric between the set of visual tokens for each of the plurality of tiles with a set of text tokens associated with the input image, the output being provided according to the similarity metric for each of the plurality of tiles.

17. A system comprising:
a processor; and
a non-transitory computer readable medium storing instructions executable by the processor, the machine-executable instructions comprising:
a text encoder that reduces a received search to a set of text tokens;
a multimodal fusion model that matches the set of text tokens to a set of visual tokens, the multimodal fusion being trained on a pretraining dataset complied from a plurality of pathology-related sources, a given training sample within the pretraining dataset comprising a pathology image and text describing the image; and
a user interface that displays an image associated with the set of visual tokens.

18. The system of claim 17, wherein the multimodal fusion model computes a similarity metric between the set of text tokens and a plurality of sets of visual tokens associated with the multimodal fusion model and matches the set of text tokens with each set of visual tokens for which the similarity metric meets a threshold value.

19. The system of claim 17, wherein the multimodal fusion model computes a similarity metric between the set of text tokens and a plurality of sets of visual tokens associated with the multimodal fusion model and matches the set of text tokens with a predetermine number of sets of visual tokens having the highest similarity metrics.

20. The system of claim 17, wherein the vision encoder is trained on a plurality of pathology images via a self-supervising learning algorithm using an objective function including a self-distillation loss and a masked image modeling loss.
